(19) United States
(12) Patent Application Publication
(10) Pub. No.: US 2022/0072267 A1
(43) Pub. Date: Mar. 10, 2022

(54) HOLOGRAPHIC SOUND FIELD DEVICE

(71) Applicant: OhmPad, Inc., Alameda, CA (US)

(72) Inventors: Amy Roither-Quintero, Alameda, CA (US); D. Robert Hoover, Bellingham, WA (US)

(73) Assignee: OhmPad, Inc., Alameda, CA (US)

(21) Appl. No.: 17/470,888

(22) Filed: Sep. 9, 2021

Related U.S. Application Data
(60) Provisional application No. 63/076,327, filed on Sep. 9, 2020.

(51) Int. Cl. A61M 21/02; H04R 1/02

(52) U.S. Cl. CPC A61M 21/02; A61M 2021/0022; H04R 1/028

(57) ABSTRACT

A holographic sound field device. The device includes a frame, one or more transducers configured to produce a holographic sound field, a controller configured to generate and transmit vibration signals to the one or more transducers. The device can be used to transmit vibrations and/or sound into a person's body to promote healing, relaxation, regeneration, and/or meditation.

### RELATED APPLICATIONS

The subject patent application claims priority to pending U.S. Provisional Patent Application No. 63/076,327 filed Sep. 9, 2020, which application is herein incorporated by reference in its entirety for all purposes.

### TECHNICAL FIELD

The present disclosure relates generally to audio technology, and more specifically to audio vibrational systems.

### DESCRIPTION OF RELATED ART

Sound interacts with the human body both by hearing through one's ears, and vibrations through the skin and bone structures. Hearing offers suggestions to the brain that can trigger neuronal, chemical, hormonal, protein and histamine stimulus to the organs and cells of the body. These influences are major factors in the health and well-being of the body. In addition, each part of the body is sensitive to vibration, both through neuronal stimulation and in toning the cells, muscles and bones. Eastern practitioners believe that Chi energy flows in the body and can be stimulated and enhanced by tactile stimulation.

Holistic, whole body health incorporates all of these processes to balance and stimulate all of the body's functions. Yoga has been able to incorporate many of these effects through thoughts and movements of the body. However, one area that yoga does not directly address is sound and vibration effects, to which the body is also very responsive.

Several technologies exist that can induce vibration into the body. These include sound beds, tuning forks, sound belts, sound vests and mechanical massage devices. However, each of these technologies presents atomistic and simplistic approaches to using vibrations as means for healing. None of these technologies provide all of these modalities in a manner robust enough to transmits broad spectrum, coherent sound fields to the body and the ears in a potent and unique way. Thus, there is a need for a flexible, holistic, standalone healing modality that utilizes vibrations in a way to stimulate healing in the body and mind through vibrations and sound, but is also flexible enough to also be used in cooperation with yoga, meditation or sleep.

### SUMMARY

The following presents a simplified summary of the disclosure in order to provide a basic understanding of certain embodiments of the present disclosure. This summary is not an extensive overview of the disclosure and it does not identify key/critical elements of the present disclosure or delineate the scope of the present disclosure. Its sole purpose is to present some concepts disclosed herein in a simplified form as a prelude to the more detailed description that is presented later.

One aspect of the present disclosure relates to a holographic sound field device. The device comprises a frame, one or more transducers configured to produce a holographic sound field, a controller configured to generate and transmit vibration signals to the one or more transducers.

In some embodiments, the device includes a top layer covering the frame and the one or more transducers, the top layer comprising material rigid enough to transmit sound accurately across the top layer and flexible enough to be comfortable against a human body. In some embodiments, the top layer further comprises material to dampen motion of the top layer in an acoustically neutral manner, in order to allow different sections of the device to have sound isolation. In some embodiments, the device includes a suspension material surrounding the one or more transducers, the suspension material configured to isolate the one or more transducers from touching the frame, wherein the suspension material is further configured to provide restoring force to support the weight of a human body. In some embodiments, the frame comprises multiple panels coupled together and a plurality of electrical and mechanical connections between the multiple panels. In some embodiments, the one or more transducers comprise exciters or tactile transducers or any other transducers that do not have a sound cone. In some embodiments, the device includes one or more amplifiers configured to provide low frequency response, low distortion, and low output impedance.

Another aspect of the present disclosure relates to a system for generating a holographic sound field. The system comprises a power source, an audio or vibrational signal source, and a pad. The pad comprises a frame, one or more transducers configured to produce a holographic sound field, and a controller configured to generate and transmit vibration signals to the one or more transducers.

Yet another aspect of the present disclosure relates to a method of healing using a holographic sound field device. The method includes laying, sitting, or standing on a pad. The pad comprises a frame, one or more transducers configured to produce a holographic sound field, and a controller configured to generate and transmit vibration signals to the one or more transducers. The method concludes with activating vibrations or sound through the pad.

These and other embodiments are described further below with reference to the figures.

### BRIEF DESCRIPTION OF THE DRAWINGS

The disclosure may best be understood by reference to the following description taken in conjunction with the accompanying drawings, which illustrate particular embodiments.

FIG. 1 illustrates a top view of a side of a holographic sound field pad, in accordance with embodiments of the present disclosure.

FIG. 2 illustrates different sections of a holographic sound field pad, in accordance with embodiments of the present disclosure.

FIG. 3 illustrates a side view of certain components of a holographic sound field pad, in accordance with embodiments of the present disclosure.

FIG. 4 illustrates an inside view of a section of a holographic sound field pad, in accordance with embodiments of the present disclosure.

FIG. 5 illustrates an example of a computer system, in accordance with one or more embodiments of the present disclosure.

### DESCRIPTION OF EXAMPLE EMBODIMENTS

Reference will now be made in detail to some specific examples of the present disclosure including the best modes contemplated by the inventors for carrying out the present disclosure. Examples of these specific embodiments are illustrated in the accompanying drawings. While the present disclosure is described in conjunction with these specific embodiments, it will be understood that it is not intended to limit the present disclosure to the described embodiments. On the contrary, it is intended to cover alternatives, modifications, and equivalents as may be included within the spirit and scope of the present disclosure as defined by the appended claims.

For example, portions of the techniques of the present disclosure will be described in the context of particular computerized systems. However, it should be noted that the techniques of the present disclosure apply to a wide variety of different computerized systems. In the following description, numerous specific details are set forth in order to provide a thorough understanding of the present disclosure. Particular example embodiments of the present disclosure may be implemented without some or all of these specific details. In other instances, well known process operations have not been described in detail in order not to unnecessarily obscure the present disclosure.

Various techniques and mechanisms of the present disclosure will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present disclosure unless otherwise noted. Furthermore, the techniques and mechanisms of the present disclosure will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

### Example Embodiments

According to various embodiments, techniques and mechanisms of the present disclosure include a suspended, semi-rigid platform with one or more transducers attached to the surface. In some embodiments, signal sources can supply audio material to the transducers which stimulates the desired effect. In some embodiments, with the use of multiple transducers and audio channels, a holographic sound field device can simulate a holographic-like sound field, which can envelop a human body and create a three dimensional (3D) sound experience for a user. A holographic sound field makes the listener feel as if they are experiencing the sound in its natural environment versus just hearing a recording of a sound. A holographic sound field is different from just a sound field produced by a speaker because a holographic sound field recreates the same phase coherency and amplitude as the actual corresponding event. This allows the ears and body to interpret the sound as if it was being heard in the natural state, such as actually being in a forest or by the ocean. By contrast, a regular sound field played by a regular speaker does not contain the same type of positioning information, which therefore prevents the listener from discerning where in space the sound originates.

All sound events create sound waves that expand through the surrounding space. A person's ears and body can sense these wave fronts. As with visual holograms, if this wave front is accurately recreated, it can produce a convincing illusion of an object, or in the case of sound, a sound event. For instance, playing the sounds of a forest through the holographic sound field device as described herein may make a listener feel like they are actually in the forest hearing the forest sounds live. In addition, the systems and methods provided create an approximation of this effect when one is lying down, sitting or and standing on the holographic sound field device. In some embodiments, the multichannel audio recording played through the holographic sound field device needs to be specially generated in order to create this effect.

According to various embodiments, he effects that can be produced depend on which implementation of the number of transducers used (e.g., anywhere from 1 to 8, or more, transducers) and their placement. Sound at different parts of body stimulates different systems of body. Sounds at the feet and legs support propulsion of the body, and also stimulate the origins of many meridians, the energetic pathways found in acupuncture, acupressure and Chinese Medicine. Sounds for the lower and central torso stimulate systems of the body such as the digestive and reproductive systems, the adrenal and pancreatic glands, and base instincts such as the need for security, identity, pleasure, and personal will. Sounds for the upper torso stimulate the respiratory, endocrine and nervous systems and the thymus and thyroid glands. Sounds at the head address the intellect, visual and audio processing and spatial sensing systems, and for some a spiritual connection. They also can stimulate the pineal and pituitary glands. The speakers at the head, when one is laying down, act almost like headphones in that they can provide stereo simulation and full frequency binaural beat tones. Transducers are placed strategically so that each body system is addressed. The holographic sound field and individual stimulus for each region of the body are two completely different effects, but they require the same hardware

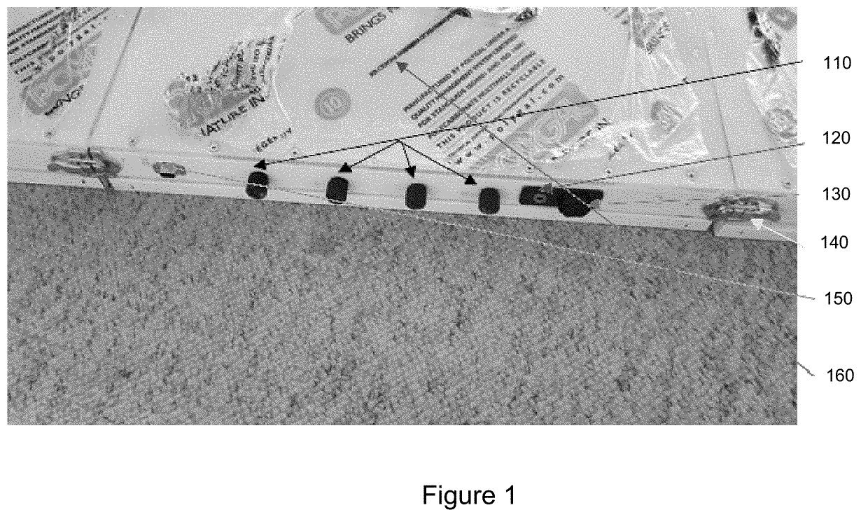

FIG. 1 illustrates a top view of a side of a holographic sound field pad, in accordance with embodiments of the present disclosure. FIG. 1 includes volume/vibration control knobs 110, power connector 120, on/off switch 130, latch 140, audio input 150 (9 pin, up to 8 channels), and top surface panel 160.

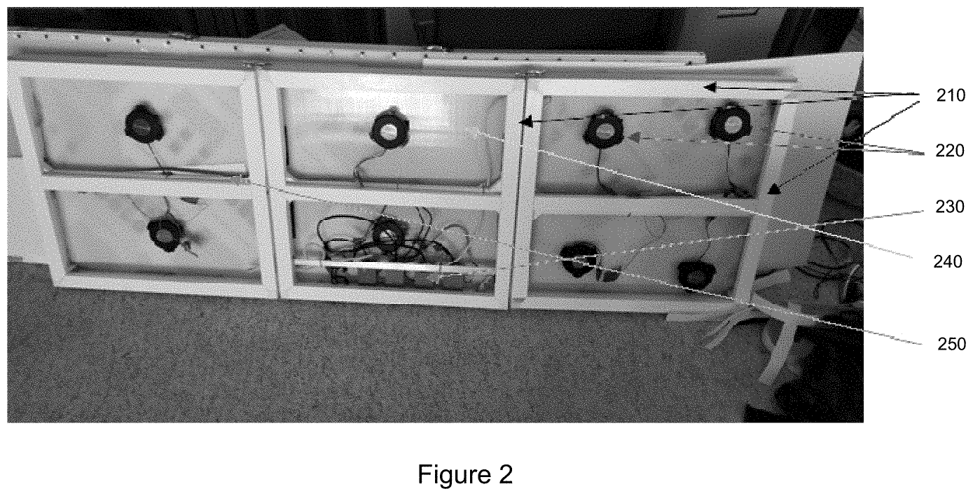

FIG. 2 illustrates different sections of a holographic sound field pad, in accordance with embodiments of the present disclosure. FIG. 2 includes frame 210, transducers 220, amplifiers 230, top surface panel 240 (as seen from underside), and suspension 250.

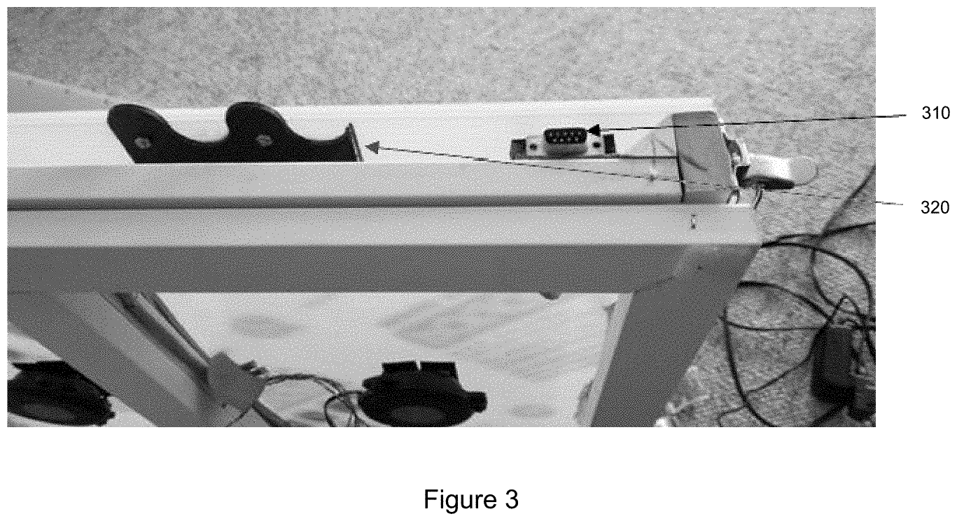

FIG. 3 illustrates a side view of certain components of a holographic sound field pad, in accordance with embodiments of the present disclosure. FIG. 3 includes interpanel audio connector 310 (9 pin) and top surface panel joiners 320.

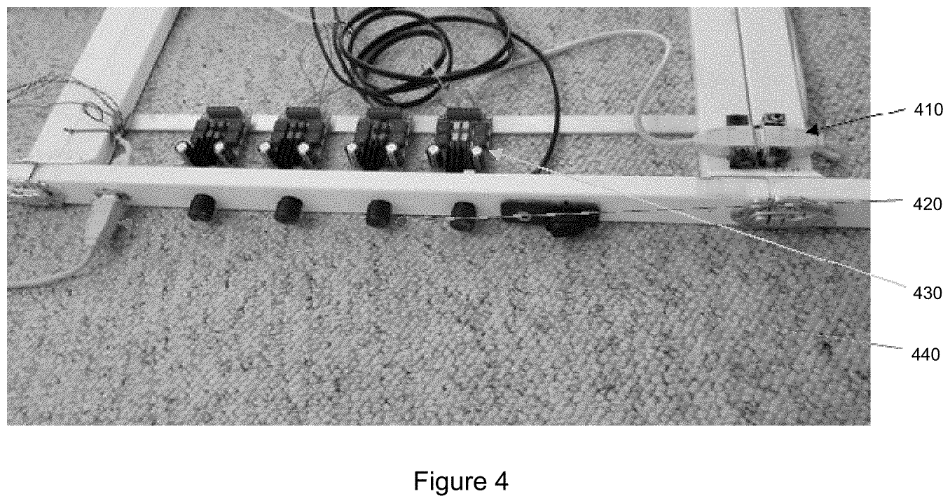

FIG. 4 illustrates an inside view of a section of a holographic sound field pad, in accordance with embodiments of the present disclosure. FIG. 4 includes interpanel audio connector 410 (9 pin), volume/vibration control knobs 430, and audio input 440 (9 pin, up to 8 channels).

According to various embodiments, the holographic sound field device comprises of a soft/hard Top Surface (160 and 240) with isolated sections driven by mono, stereo or multichannel sound sources. In some embodiments, the primary suspension of the Top Surface is done by developing a tension between the Top Surface and the outside walls of the frame (210). In some embodiments, with this “stretching” of the top surface and the bending of the frame, a spring like restoring force gives a softer feel to the bendable hard surface when it is stood on or laid on. In some embodiments, the restoring force is strong enough to support a large person but there is still a slight softening bending to the surface. In addition, in some embodiments, the Top Surface can transmit phase accurate full frequency sounds from the subsonic to ultrasonic ranges (10 Hz to 20,000 Hz).

In some embodiments, the Top Surface is also supported by vibration absorbing suspension blocks, or walls, in the middle and/or the edges of the panels. In such embodiments, these connect between the Top Surface and the bottom of the Frame and they provide even more spring like restoring forces and add solidity to the surface. In some embodiments, these absorbing supports are also used to isolate the different channels of vibration which gives better multichannel stereo effects. In some embodiments, the absorbing supports provide enough restoring force for a heavy person to stand on the holographic sound field device. In such embodiments, the tension needs to be extremely high to keep material from bending too much, because if the material bends too much, the transducers are pushed into the bottom of the frame, which prevents the transducers from moving.

According to various embodiments, the Top Surface can comprise one, two, three or more sections. In some embodiments, for each section, there is a surface, a frame, electrical and/or mechanical connections (310) that form a linking system between sections. In some embodiments, there are also interlocking connecting “fingers” (320) added to the adjacent Top Surfaces to couple energy and effectively remove the seam between the multiple Top Surfaces. In some embodiments, the different sections are for isolation as well as ease for shipping and storing. In such embodiments, panels can be attached by fixed or separable hinges as well as by latches to connect the surfaces. In some embodiments, the panels are isolated so that they move separately even though they are connected mechanically. In one embodiment, the same sounds are played top to bottom. In an alternative embodiment, different panels play different sounds.

In some embodiments, because the Top Surface is essentially one radiating panel and the driving transducers can all be the same, the sound/vibration emanating from the panel can be phase coherent and full bandwidth. Phase coherence occurs when all the vibrational frequencies are produced at the same time and full bandwidth means all the audible frequencies are reproduced. In some embodiments, with a large number of transducers, the vibrational power available can be considerable. When properly designed, a system can also have very low distortion. As a result, the fidelity of the system can seem to be better than many home “Hi-Fi” systems. The illusion of being inside a 3D holographic sound field can be convincing. For instance, it might be desirable that a person feels that they are in an outdoor garden. With the inclusion of some distributed bird sounds and the rustling of tree leaves properly presented, it might generate a calming feeling in the listener. In some embodiments, the sound field is a variable shape depending on what frequency is being played. High frequencies are emitted as a beam of sound and low frequencies radiate out in all directions. In some embodiments, the size is 2 feet by 6 feet wide and 8 to 10 feet high, if standing. In some embodiments, when laying down, the sound is absorbed by the body so it does not need to go as high.

In current technology, typical “sound bed” systems have speakers mounted below a mattress or cushion. This approach has a few drawbacks. For example, the mattress or cushion is not stable enough to do Yoga, Tai Chi, Qi Gong or other balance oriented practices on. In addition, the cushion normally only passes low frequencies to the mass of the body, eliminating a large range of healing and energy effects and modalities available from higher frequencies. In addition, any stereo effects are also muted by such a covering. The techniques and mechanisms disclosed herein address these drawbacks. The following paragraphs provide a detailed description of important features of a holographic sound field device, in accordance with embodiments of the present disclosure.

Top Surface (160)

In some embodiments, the Top Surface is a very critical component. According to various embodiments, it has to be strong, rigid, flexible, lightweight and acoustically neutral. More specifically, the strength is needed for it to be robust with both heavy and diminutive users while standing, dancing, sitting or lying down.

In some embodiments, the material needs to be rigid to transmit the sound accurately over the whole surface. The more rigid the material is the more accurate the performance will be. In some embodiments, the material has to be flexible so that it is not brittle and so that it is comfortable to stand, sit and lie on.

In some embodiments, the mass of the material will determine how much power needs to be applied to move the material quickly. In some embodiments, the surface partially acts like a speaker cone. The lighter the material the clearer the sound.

All materials have an acoustic signature. For example, a surface made of rubber would produce a muffled sound, and one made of tin would provide a clangy sound. However, this needs to be minimized so that the emanated acoustic field is accurate. There are many materials that can be used to achieve these ends. According to various embodiments, it is possible to use specially designed fiberglass compounds, carbon fiber-based surfaces, aluminum hybrid materials and various forms of plastic compounds. In some embodiments, extruded polycarbonate cellular honeycomb panels are preferred. This is because polycarbonate is incredibly strong and flexible. In such embodiments, the cellular function dramatically reduces the weight without diminishing its strength and durability. Acoustically, it can be is quite neutral.

According to various embodiments, by selectively removing material from the panel, it is possible to have flexible sections that can be used for acoustic suspensions, or islands, and to produce bendable creases for folding and structural elements. In some embodiments, it is possible to machine a single piece of this material to make the surface, frame and reinforcements. This allows for low cost semi-automated fabrication.

Frame (210)

According to various embodiments, the frame is also a critical element to the proper operation of the holographic sound field device. In some embodiments, it needs to withstand the strong horizontal forces coming from the leveraged pull of the Top Surface when someone is standing or laying on the surface. In some cases, this force can be many times what the weight of the person that the surface is supporting

In some embodiments, a Frame can be split into multiple pieces that can be dissembled or joined together. In such embodiments, this feature allows for convenient carrying, shipping and storage. There are several possible configurations, depending on the cost, fidelity and performance needed. The current implementation consists of three 2′×2′ panels with electrical and mechanical connections between them and locking latches. The central panel has the amplifiers and associated electronics.

Suspension (250)

In some embodiments, a soft flexible material is used for two functions. One is to supply some of the restoring force needed for the Top Surface to support standing, sitting or laying down, and also to isolate and dampen each section of the holographic sound field device. These also limit the travel of the Top Surface to keep the Transducers from hitting the bottom of the Frame. The resulting isolation allows for the multichannel holographic sound field function to work correctly and gives a uniform support across the holographic sound field device.

Panel Coupling

In some embodiments, the connection between top surface panels can be a soft or a rigid connection. In some embodiments, the panels need to be joined close enough that it does not pinch a person's body or clothing. In some embodiments, flexible fingers are used between the panels. This provides more isolation between the panels so the sound does not couple as easily across. In addition, this enables the multichannel effects to be more clearly pronounced. In applications for stereo or mono effects, for instance, if using only one or two transducers, the coupling between panels needs to be much stronger so that the sound is uniform across the whole surface. This be can be accomplished by using stiffer fingers, connecting rods, and ribs (ribs make the top surface more rigid).

Damping

As mentioned, the suspension material is used for isolating the sections of the holographic sound field device. This is done by dampening the motion of the top surface. In some embodiments, this effect needs to be acoustically neutral. Careful selection of this material is important. In some embodiments, both rubber and polyurethane foams of different densities are used. In some embodiments, the isolation is needed for spatial resolution features. Isolation is good for getting good separation to create holographic sound field. Each piece needs to be independent so that it is not creating a mono effect. Isolation is also required to create binaural beat frequencies, which require 2 distinct sounds.

According to various embodiments, damping has four functions: suspension, isolation, surface resonance reduction, and cushioning. In some embodiments, suspension provides a restoring force that keeps the person from bottoming out when standing, sitting or lying on the holographic sound field device. It also protects the transducers from being pushed into the bottom panel and preventing them from performing properly. In some embodiments, isolation separates the panels into left and right channels for multichannel separation. It can also be placed where panels join to give panels separation from each other. The top surface, transducers, and the human body all have unique natural resonant frequencies. In some embodiments, surface resonance reduction, or damping, reduces the resonances so it improves the accuracy of the sound reproduction. In some embodiments, cushioning gives a feeling of support and softness that is different from the tensioning of the surface.

Transducers (220)

There are many types of transducers available that produce vibration but not necessarily sound. In some embodiments, there are two basic types that work well for a holographic sound field device: exciters and tactile transducers.

Exciters are essentially a loudspeaker without a cone that is normally used for sound radiation. In some embodiments, these are attached to a Top Surface with a mount directly attached to the voice coil/coils. In such embodiments, the magnet structure provides a mass that the voice coil can push against. The reactive force goes directly into the surface that it is mounted to.

Tactile transducers are more self-contained. In some embodiments, the voice coil, magnet and mount are integrated into a sealed enclosure. In such embodiments, the reactive force is transmitted through the enclosure. These transducers are generally more robust but can have some performance limitations. Most notably, in the frequency extremes. The extra mass of the case can slow their response, the stiffness of the mount inside the case limits the high frequency compliance.

Depending on the cost, performance, durability and size requirements either of these designs can be used in the holographic sound field device. Other types of transducers have a sound cone, which is what radiates the sound in most speakers. The transducers and exciters have no speaker cone and use the Top Surface for radiating sound. In various embodiments of the present disclosure, the sound cone is the surface.

Amplifiers (430)

According to various embodiments, depending on the number of channels desired, single or multiple power amplifiers can be integrated in to the holographic sound field device. In some embodiments, a standard multichannel system can have one, two, four, six, or eight channels. In some embodiments, the panels are sectioned off for the stimulation of desired sections of the body. In some embodiments, a standard arrangement for an 8 channel system, with three panels, could have two sections for the upper panel. In such embodiments, there would be one for the head and a second for the shoulders. The mid panel could have two or four transducers and a two channel amplifier. The foot section could have two transducers and a two channel amplifier.

In some embodiments, 50 Watt Class-D switching amplifiers (430) are used. In such embodiments, the amplifiers are small, inexpensive and have very low heat dissipation. In some embodiments, each stereo amplifier has its own volume control knob (420) on the outside of the frame. Different transducers require and handle different amounts of power. In some embodiments, the transducers are 10 w each. In some embodiments, each amplifier board is capable of 50 w per channel. According to various embodiments, the necessary qualities in the amplifiers are: low frequency response, low distortion, and low output impedance.

Signal Sources

According to various embodiments, sourcing signals can come from a number of means. In some embodiments, the sourcing electronics are external to the holographic sound field device. In some embodiments, a female 9-pin D-type connector providing 8 channels of audio is used. In such embodiments, with adapters, this can be connected to a 7.1 surround, stereo or mono line level audio source. For instance, this can be sourced from home audio equipment, computers, tablets, cell phones and Bluetooth and similar radio links. These interfacing electronics, e.g., Ethernet and WiFi broadcasting, can provide remote media servers reception. In some embodiments, these can be external or be added inside the holographic sound field device. In some embodiments, internal media servers utilize local flash storage, SD or USB thumb drives along with internal sound and music synthesis technology that can be added to make a complete stand-alone media system. In some embodiments, external computers, tablets and phone applications can be used to control these systems.

Power Sources

According to various embodiments, an external 12 VDC 10 A line powered switching power supply is utilized. In some embodiments, the minimum power requirement is 80 watts, and the maximum is 400 watts. In some embodiments, it utilizes a standard barrel connector to transmit power. In some embodiments, a frame mounted rocker switch (130) with an integrated LED controls the power. In some embodiments, external batteries can also be plugged into this power jack for remote operation.

Auto Equalization

According to various embodiments, the holographic sound field device utilizes sensors to tune the equalization of the pad for specific state that the device is in at any given moment, whether a person is standing on the mat or lying down on it.

Final Fabrication:

In some embodiments, the option exists to have the whole holographic sound field device made out of polycarbonate that wraps around to create the frame. In such embodiments, the device would have lower weight and lower cost to have the frame and surface integrated into a single unit, manufactured as a single unit. In such embodiments, it is possible to route the material of the Top Surface to make the bends.

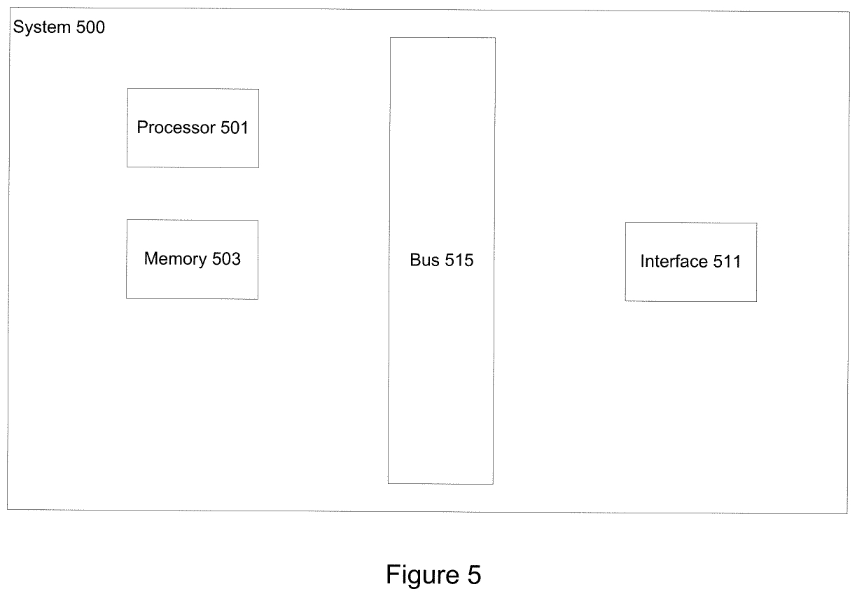

FIG. 5 illustrates one example of a computer system 500. System 500 can be used to implement any electronic or computing functions in the systems and mechanisms disclosed herein. For example, system 500 can be used to implement any of the software for a holographic sound field pad, as well as implementing any of the software for controlling and managing any portions of the holographic sound field pad. According to particular embodiments, a system 500 suitable for implementing particular embodiments of the present disclosure includes a processor 501, a memory 503, an interface 511, and a bus 515 (e.g., a PCI bus or other interconnection fabric). When acting under the control of appropriate software or firmware, the processor 501 is responsible for implementing applications such as an operating system kernel, a storage driver, and one or more applications. Various specially configured devices can also be used in place of a processor 501 or in addition to processor 501. The interface 511 is typically configured to send and receive data packets or data segments over a network.

Particular examples of interfaces supported include Ethernet interfaces, frame relay interfaces, WIFI interfaces, Bluetooth interfaces, cable interfaces, DSL interfaces, token ring interfaces, and the like. In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control communications-intensive tasks such as packet switching, media control and management.

According to various embodiments, the system 500 is a system configured to operate and manage any of the systems or subsystems described herein. For example, the system 500 may be configured execute the methods disclosed herein. The system may include one or more hardware elements as shown in FIG. 5. In some implementations, one or more of the system components may be virtualized. For example, a physical server may be configured in a localized or cloud environment. The physical server may implement one or more virtual server environments in which the algorithm engine is executed. Although a particular system is described, it should be recognized that a variety of alternative configurations are possible. For example, the modules may be implemented on another device connected to one or multiple servers.

In the foregoing specification, the present disclosure has been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of present disclosure.

What is claimed is:

1. A holographic sound field device, the device comprising:
a frame;
one or more transducers configured to produce a holographic sound field; and
a controller configured to generate and transmit vibration signals to the one or more transducers.

2. The device recited in claim 1, further comprising a top layer covering the frame and the one or more transducers, the top layer comprising material rigid enough to transmit sound accurately across the top layer and flexible enough to be comfortable against a human body.

3. The device recited in claim 2, wherein the top layer further comprises material to dampen motion of the top layer in an acoustically neutral manner, in order to allow different sections of the device to have sound isolation.

4. The device recited in claim 1, further comprising a suspension material surrounding the one or more transducers, the suspension material configured to isolate the one or more transducers from touching the frame, wherein the suspension material is further configured to provide restoring force to support the weight of a human body.

5. The device recited in claim 1, wherein the frame comprises multiple panels coupled together and a plurality of electrical and mechanical connections between the multiple panels.

6. The device recited in claim 1, wherein the one or more transducers comprise exciters or tactile transducers or any other transducers that do not have a sound cone.

7. The device recited in claim 1, further comprising one or more amplifiers configured to provide low frequency response, low distortion, and low output impedance.

8. A system for generating a holographic sound field, the system comprising:
a power source;
an audio or vibrational signal source; and
a pad, the pad comprising:
a frame;
one or more transducers configured to produce a holographic sound field; and
a controller configured to generate and transmit vibration signals to the one or more transducers.

9. The system recited in claim 8, wherein the pad further comprises a top layer covering the frame and the one or more transducers, the top layer comprising material rigid enough to transmit sound accurately across the top layer and flexible enough to be comfortable against a human body.

10. The system recited in claim 9, wherein the top layer further comprises material to dampen motion of the top layer in an acoustically neutral manner, in order to allow different sections of the device to have sound isolation.

11. The system recited in claim 8, wherein the pad further comprises a suspension material surrounding the one or more transducers, the suspension material configured to isolate the one or more transducers from touching the frame, wherein the suspension material is further configured to provide restoring force to support the weight of a human body.

12. The system recited in claim 8, wherein the frame comprises multiple panels coupled together and a plurality of electrical and mechanical connections between the multiple panels.

13. The system recited in claim 8, wherein the one or more transducers comprise exciters or tactile transducers or any other transducers that do not have a sound cone.

14. The system recited in claim 8, wherein the pad further comprises one or more amplifiers configured to provide low frequency response, low distortion, and low output impedance.

15. A method for healing using a holographic sound field device, the method comprising:
laying, sitting, or standing on a pad, the pad comprising:
a frame;
one or more transducers configured to produce a holographic sound field; and
a controller configured to generate and transmit vibration signals to the one or more transducers; and
activating vibrations or sound through the pad.

16. The method recited in claim 15, wherein the pad further comprises a top layer covering the frame and the one or more transducers, the top layer comprising material rigid enough to transmit sound accurately across the top layer and flexible enough to be comfortable against a human body.

17. The method recited in claim 16, wherein the top layer further comprises material to dampen motion of the top layer in an acoustically neutral manner, in order to allow different sections of the device to have sound isolation.

18. The method recited in claim 15, wherein the pad further comprises a suspension material surrounding the one or more transducers, the suspension material configured to isolate the one or more transducers from touching the frame, wherein the suspension material is further configured to provide restoring force to support the weight of a human body.

19. The method recited in claim 15, wherein the frame comprises multiple panels coupled together and a plurality of electrical and mechanical connections between the multiple panels.

20. The method recited in claim 15, wherein the one or more transducers comprise exciters or tactile transducers or any other transducers that do not have a sound cone.
